SEQUENCE LISTING (6 sequences)

SEQ ID NO: 1 - DNA, 18 bp, synthetic construct
  source 1..18: mol_type = other DNA
taggcaccagggtgtgat

SEQ ID NO: 2 - DNA, 16 bp, synthetic construct
  source 1..16: mol_type = other DNA
ctcctcaggggccaca

SEQ ID NO: 3 - DNA, 23 bp, synthetic construct
  source 1..23: mol_type = other DNA
gacgaagagcccttacagttcca

SEQ ID NO: 4 - DNA, 20 bp, synthetic construct
  source 1..20: mol_type = other DNA
tctgcagtgcctccactatg

SEQ ID NO: 5 - DNA, 19 bp, synthetic construct
  source 1..19: mol_type = other DNA
cctggtccctctggaaatg

SEQ ID NO: 6 - DNA, 20 bp, synthetic construct
  source 1..20: mol_type = other DNA
ggaagcctctttctcctctc

(19) United States
(12) Patent Application Publication
(10) Pub. No.: US 2024/0041814 A1
(43) Pub. Date: Feb. 8, 2024

(54) USE OF REAGENT FOR DETECTING RETINOL METABOLITE IN PREPARATION OF TOOL FOR DIAGNOSING AND/OR TREATING PNEUMOCONIOSIS

(71) Applicant: Institute of Basic Medical Sciences of Chinese Academy of Medical Sciences, Beijing (CN)

(72) Inventors: Jing WANG, Beijing (CN); Meiyue SONG, Beijing (CN); Jiaxin WANG, Beijing (CN)

(73) Assignee: Institute of Basic Medical Sciences of Chinese Academy of Medical Sciences, Beijing (CN)

(21) Appl. No.: 18/156,421

(22) Filed: Jan. 19, 2023

(30) Foreign Application Priority Data
Aug. 8, 2022 (CN) 202210944602.7

(51) Int. Cl. A61K 31/203; A61P 11/00; G01N 30/72

(52) U.S. Cl. CPC A61K 31/203; A61P 11/00; G01N 30/7233

(57) ABSTRACT

The present disclosure provides use of a reagent for detecting retinol metabolites in preparation of a tool for diagnosing and/or treating pneumoconiosis, and relates to the technical field of biomedicine. The present disclosure verifies that retinol metabolism disturbance and all-trans retinoic acid (ATRA) deficiency are closely related to the pathogenesis of pneumoconiosis. Therefore, the present disclosure sets forth use of retinol and ATRA as biomarkers for screening the pneumoconiosis and use thereof in preparation of products related to the diagnosis of the pneumoconiosis; meanwhile, intervention of the pneumoconiosis with the ATRA can effectively delay the progression of the pneumoconiosis. Therefore, the present disclosure first sets forth use of the ATRA as a medicament for treating the pneumoconiosis.

### CROSS REFERENCE TO RELATED APPLICATION

This application claims the benefit and priority of Chinese Patent Application No. 202210944602.7, filed on Aug. 8, 2022; the disclosure of which is incorporated herein by reference in its entirety as part of the present application.

### REFERENCE TO SEQUENCE LISTING

A computer readable XML file entitled “SEQUENCE LISTING.XML”, that was created on Nov. 28, 2022, with a file size of 7 kb, contains the sequence listing for this application, has been filed with this application, and is hereby incorporated by reference in its entirety.

### TECHNICAL FIELD

The present disclosure relates to the technical field of biomedicine, in particular to use of a reagent for detecting retinol metabolites in preparation of a tool for diagnosing and/or treating pneumoconiosis.

### BACKGROUND

Pneumoconiosis is an occupational pulmonary disease manifesting as diffuse pulmonary fibrosis, caused by long-term inhalation of pathogenic dust. Clinical manifestations are cough, expectoration, chest distress, chest pain, and dyspnea, and pathological changes are characterized by pulmonary inflammation and diffuse nodular fibrosis. Even if dusty environment is removed, disease progression cannot be prevented, and finally it may be possible to develop respiratory failure and even death. Pneumoconiosis is common in those who engage in contact cutting, polishing, and stone grinding, including quarrying, mining, coal mining, sandblasting, potting, stone masonry, road construction, rock drilling, tunnel operation, and jewelry polishing.

Pneumoconiosis is one of the most important occupational diseases in the world. To date, due to unclear pathogenesis of pneumoconiosis, there is no effective therapy. Lung transplantation remains the final choice of patients with pneumoconiosis complicated with respiratory failure. At present, in view of pneumoconiosis, there is no therapeutic regimen for improving prognosis and lung function, but only symptomatic treatment measures including cough-suppressing, phlegm-transforming, and improvement of shortness of breath and dyspnea can be taken; in necessity, anti-inflammatory therapy may be conducted.

All-trans retinoic acid (ATRA) is an active metabolite of vitamin A. ATRA can regulate genes related to cell growth, proliferation, differentiation, and apoptosis. It is a differentiation inducer that is first used clinically. It is perceived as inducing abnormally accumulative promyelocytes to differentiate into mature granulocytes and further inducing cell death and inhibiting proliferation. It is the drug of first choice to treat acute promyelocytic leukemia at present. However, the therapeutic effect of the ATRA on fibrosis of pneumoconiosis remains unclear.

### SUMMARY

In view of this, an objective of the present disclosure is to provide use of a reagent for detecting retinol metabolites in preparation of a tool for diagnosing and/or treating pneumoconiosis. Retinol and ATRA are used as biomarkers for screening pneumoconiosis, and the ATRA is used as a medicament for treating pneumoconiosis, so that a significant therapeutic effect is obtained.

To achieve the above objective, the present disclosure provides the following technical solutions:

The present disclosure provides use of a reagent for detecting retinol metabolites in preparation of a tool for diagnosing pneumoconiosis.

Preferably, the retinol metabolites may include one or more of ATRA, a precursor of the ATRA, a metabolite of the ATRA, and a modified product of the ATRA.

Preferably, detection methods of the retinol metabolites may include metabonomics or targeted high-performance liquid chromatography-tandem mass spectrometry (HPLC-MS/MS).

Preferably, a sample for detecting the retinol metabolites may include lung tissue.

The present disclosure further provides use of one or a combination of more selected from the group consisting of ATRA, a precursor of the ATRA, a metabolite of the ATRA, and a modified product of the ATRA in preparation of a medicament for treating pneumoconiosis.

Preferably, the pneumoconiosis may include silicosis, coal worker's pneumoconiosis, graphite pneumoconiosis, carbon black pneumoconiosis, asbestosis, talc pneumoconiosis, cement pneumoconiosis, mica pneumoconiosis, kaolin pneumoconiosis, aluminosis, arc-welders pneumoconiosis, and foundry workers' pneumoconiosis.

Beneficial effects: The present disclosure provides use of a reagent for detecting retinol metabolites in preparation of a tool for diagnosing pneumoconiosis. It is found in the examples that retinol metabolism disturbance and ATRA deficiency are closely related to the pathogenesis of pneumoconiosis. Therefore, the present disclosure sets forth use of retinol and ATRA as biomarkers for screening the pneumoconiosis and use thereof in preparation of products related to the diagnosis of the pneumoconiosis; meanwhile, intervention of the pneumoconiosis with the ATRA can effectively delay the progression of the pneumoconiosis. Therefore, the present disclosure first sets forth use of the ATRA as a medicament for treating the pneumoconiosis.

### BRIEF DESCRIPTION OF THE DRAWINGS

FIG. 1A-C illustrate a relationship of malfunction of retinol metabolic pathway in the pneumoconiosis tissue with ATRA deficiency, where panels, FIGS. 1A and 1B represent a volcano plot of differences in metabolites between PBS group and silica group, and a KEGG pathway map; panel FIG. 1C illustrates the detection of the content of ATRA in lung tissues of mice of the PBS group and the silica group; ***P<0.001;

FIG. 2A-K illustrates the effect of ATRA on pneumoconiosis, where panel A is a schematic diagram showing an experiment for ATRA treatment; panels B and C illustrate mouse lung function, where IC represents inspiratory capacity and Cst represents quasi-static compliance; panel D illustrates mouse right ventricular function, where RVSP represents right ventricular systolic pressure; panel E illustrates mouse HE staining; panels F and G illustrates concentrations of IL-1β and IL-6 in mouse bronchoalveolar lavage fluid, where BALF represents the bronchoalveolar lavage fluid; panel H illustrates mouse Masson staining; panels I and J illustrates translational and transcriptional levels of Fn-1 in mouse lung tissues; panel K illustrates the transcriptional level of Col-I in mouse lung tissues; **P<0.01, ***P<0.001; and

FIG. 3A-K illustrates the effect of vitamin A deficiency (VAD) on pneumoconiosis, where panel A is a schematic diagram showing an evaluation of the effect of ATRA deficiency induced by VAD diet on mice with pneumoconiosis; panels B and C illustrate mouse lung function; panel D illustrates mouse right ventricular function, where RVSP represents right ventricular systolic pressure; panel E illustrates mouse HE staining; panels F and G illustrates concentrations of IL-1β and IL-6 in mouse BALF; panel H illustrates mouse Masson staining; panels I and J illustrates translational and transcriptional levels of Fn-1 in mouse lung tissues; panel K illustrates the transcriptional level of Col-I in mouse lung tissues; **P<0.01, ***P<0.001.

### DETAILED DESCRIPTION OF THE EMBODIMENTS

The present disclosure provides use of a reagent for detecting retinol metabolites in preparation of a tool for diagnosing pneumoconiosis.

In the present disclosure, the retinol metabolites may preferably include ATRA, a precursor of the ATRA, a metabolite of the ATRA, and a modified product of the ATRA. In the present disclosure, samples for detecting the retinol metabolites may preferably include lung tissue. In the present disclosure, detection methods of the retinol metabolites may preferably include metabonomics or targeted HPLC-MS/MS.

In the present disclosure, the pneumoconiosis may preferably include types of the pneumoconiosis diagnosed according to the “diagnostic criteria for pneumoconiosis” and “pathological diagnostic criteria for pneumoconiosis”, and more preferably include silicosis, coal worker's pneumoconiosis, graphite pneumoconiosis, carbon black pneumoconiosis, asbestosis, talc pneumoconiosis, cement pneumoconiosis, mica pneumoconiosis, kaolin pneumoconiosis, aluminosis, arc-welders pneumoconiosis, and foundry workers' pneumoconiosis. In the examples of the present disclosure, treatment of the mice with pneumoconiosis with ATRA can significantly improve cardiopulmonary function, inflammation, and fibrosis thereof. Compared with control mice, IC and Cst of ATRA-treated mice with pneumoconiosis are improved to a certain extent, and RVSP is further lowered to a certain extent. Pathological staining shows that the ATRA can reduce the severity of inflammation and fibrosis. ATRA can further lower concentrations of inflammatory factors IL-1β and IL-6 in the BALF of the mice with pneumoconiosis and reduce levels of fibrosis factors FN-1 and Col-I. Therefore, the present disclosure first sets forth use of the ATRA as a medicament for treating pneumoconiosis.

In the examples of the present disclosure, mice with pneumoconiosis are used for experiments. Compared with control mice, the mice with pneumoconiosis have pulmonary metabolic abnormalities, and a large number of metabolites significantly increase or decrease. Abnormal metabolic pathways occurred in pneumoconiosis mainly include tryptophan metabolism, steroid hormone metabolism, and retinol metabolism. Active retinol metabolite ATRA in lung tissues of the mice with pneumoconiosis is significantly lower than that in the control mice. After that, the mice with pneumoconiosis are intervened with a vitamin A-deficient (VAD) diet to consume in vivo ATRA and aggravate the progression of pneumoconiosis; compared with a vitamin A-sufficient (VAS) group fed with a normal diet, cardiopulmonary functions of the mice with pneumoconiosis in the VAD group are aggravated to varying degrees. Pathological staining further indicates that ATRA deficiency aggravates the severity of inflammation and fibrosis in the mice with pneumoconiosis. Inflammatory factors, IL-1β and IL-6, as well as fibrosis factors, FN-1 and Col-I, further increase in the mice with pneumoconiosis due to ATRA deficiency. It has thus been demonstrated that retinol metabolites, particularly the ATRA, can be used as detection markers for pneumoconiosis.

The present disclosure further provides use of one or a combination of more selected from the group consisting of ATRA, a precursor of the ATRA, a metabolite of the ATRA, and a modified product of the ATRA in preparation of a medicament for treating pneumoconiosis.

The use of a reagent for detecting retinol metabolites in preparation of a tool for diagnosing and/or treating pneumoconiosis provided by the present disclosure will be described in detail below in conjunction with examples, but they should not be construed as limiting the protection scope of the present disclosure.

### Example 1

Pneumoconiosis-induced retinol metabolism disturbance led to ATRA deficiency.

1.1 Construction of a Mouse Model of Pneumoconiosis and Metabolomic Detection.

Male C57BL/6J mice (aged 8-10 weeks and weighing 25-30 g) were selected and housed in an SPF grade laboratory animal room. The mice were randomly divided into two groups: (1) PBS group (n=10); and silica group (n=15). The model of pneumoconiosis was constructed by one-time intratracheal instillation of silica, and the control group was administered with an equal amount of PBS. Six weeks after modeling, mouse lung tissues were collected for non-target metabolomic detection based on HPLC-MS/MS. According to the result of non-target metabolomics analysis, the mouse model of pneumoconiosis was constructed, and the content of ATRA in lung tissues of mice with pneumoconiosis and control mice was detected quantitatively in a targeted manner by HPLC-MS/MS.

1.2 Interpretation of Results

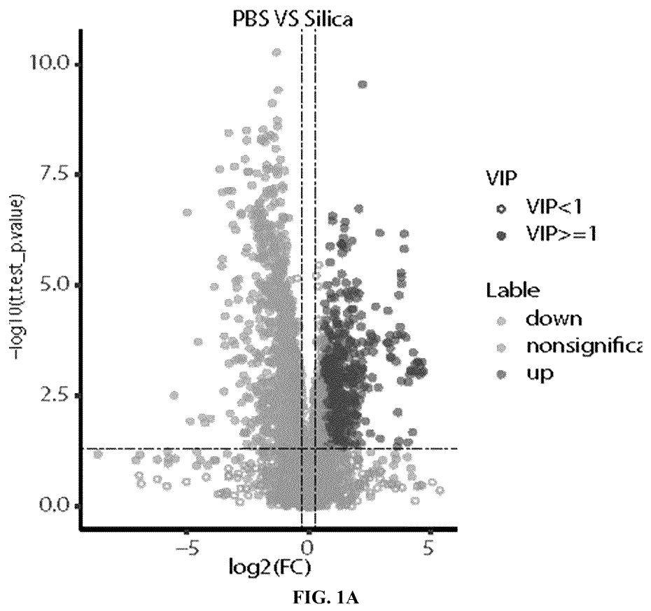

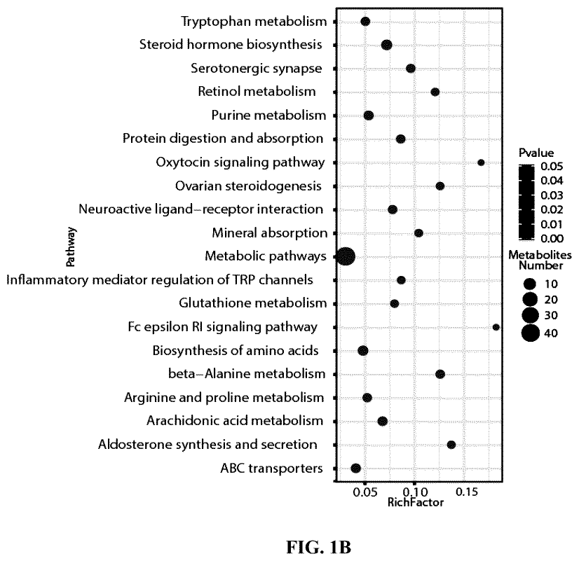

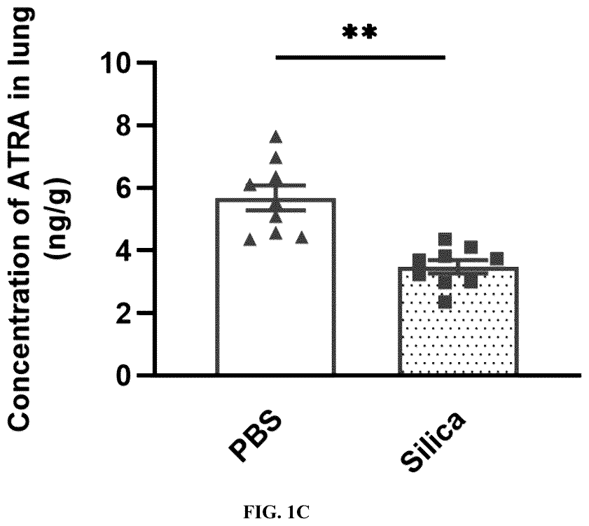

Compared with control mice, the mice with pneumoconiosis had pulmonary metabolic abnormalities, and a large number of metabolites significantly increased or decreased (FIG. 1A). Abnormal metabolic pathways occurred in pneumoconiosis mainly included tryptophan metabolism, steroid hormone metabolism, and retinol metabolism (FIG. 1B). Active retinol metabolite ATRA in lung tissues of the mice with pneumoconiosis was significantly lower than that in the control mice (FIG. 1C).

The above descriptions are merely preferred implementations of the present disclosure. It should be noted that a person of ordinary skill in the art may further make several improvements and modifications without departing from the principle of the present disclosure, but such improvements and modifications should be deemed as falling within the protection scope of the present disclosure.

### Example 2

ATRA Treated Pneumoconiosis Effectively.

2.1 Construction of a Mouse Model of Pneumoconiosis and Administration of ATRA

Male C57BL/6J mice (aged 8-10 weeks and weighing 25-30 g) were selected and housed in an SPF grade laboratory animal room, and the model of pneumoconiosis was constructed by one-time intratracheal instillation of silica (Si); the mice were divided into four groups (n=9): (1) PBS+vehicle group: 40 μL of sterile phosphate-buffered saline (PBS) was administered by intratracheal instillation; two weeks later, corn oil was administered intragastrically 3 times a week, for 4 weeks; (2) PBS+ATRA group: 40 μL of sterile PBS was administered by intratracheal instillation; two weeks later, 10 mg/kg ATRA was administered intragastrically 3 times a week, for 4 weeks; (3) Si+vehicle group: a silica suspension (300 mg/mL, 40 μL) was administered by intratracheal instillation; two weeks later, corn oil was administered intragastrically 3 times a week, for 4 weeks; (4) Si+ATRA group: a silica suspension (300 mg/mL, 40 μL) was administered by intratracheal instillation; two weeks later, 10 mg/kg ATRA was administered intragastrically 3 times a week, for 4 weeks. All mice were sacrificed after 6 weeks, and the corresponding samples were collected for detection.

2.2 Cardiopulmonary Function Test

An anesthetized mouse was fixed on an experimental bench, and an 8 gauge needle connected with a high-fidelity pressure sensor was inserted into the right ventricle to directly measure the invasive right ventricular systolic pressure (RVSP). The RVSP was recorded and analyzed by a Power Lab data acquisition and analysis system (PL 3504, AD Instruments, Australia). Mouse lung function was detected by a pulmonary function testing system (DSI Buxco, USA). Before experiment, the mouse was anesthetized by intraperitoneal injection of 0.4 mL/100 g 2% pentobarbital, tracheotomy was performed, a trachea cannula was inserted, and a ventilator was connected. Next, FRC, PV, FV, and RC were automatically tested by a PET system. A statistical analysis was conducted on indexes closely related to lung function changes of pneumoconiosis, including inspiratory capacity (IC) and quasi-static compliance (Cst).

2.3 Pathological Staining

The left lung was fixed in 4% paraformaldehyde for 24 h, dehydrated, paraffin-embedded, sectioned (to 5 μm), and subjected to HE staining and Masson staining, respectively. For HE staining, inflammation was scored based on Szapiel scores; for Masson staining, the severity of fibrosis was scored by Aschroft scores. The sections were scanned, photographed and counted by a 3D HISTECH digital slide scanner.

2.4 Enzyme-Linked Immunosorbent Assay (ELISA)

Concentrations of inflammatory factors IL-1β and IL-6 in mouse BALF were detected by using ELISA kits.

2.5 Western Blot

Lung tissue proteins of all mice were extracted. A 10% polyacrylamide gel was prepared, and 10 μg of protein was loaded and electrophoresed at 80 V for 30 min; after the voltage was adjusted to 120 V, the electrophoresis was continued until it ended. Polyvinylidene fluoride (PVDF) membrane was used for the transfer and blocked with 5% skimmed milk powder for 1 h, the primary antibody FN-1 (abcam, 1:1,000) was incubated at 4° C. overnight and the secondary antibody was incubated at room temperature for 1 h, and finally blots were developed by chemiluminescence. Then, the band was incubated with (3-actin antibody. The remaining steps were identical as described above.

2.6 qPCR

Lung tissue RNA of all mice was extracted; cDNA was obtained by using a reverse transcription kit (KR103, Tiangen Biotechnology, Beijing, China); qPCR was conducted by using a SYBR Green I Q-PCR Kit (TransGen Biotech, Beijing China); data collection and analysis were conducted by Bio-Rad IQ5 system.

TABLE 1
Primer sequences (5′ to 3′)

| | | | |
|---|---|---|---|
| ß-actin | F | TAGGCACCAGGGTGTGAT | SEQ ID NO. 1 |
| | R | CTCCTCAGGGGCCACA | SEQ ID NO. 2 |
| Fn-1 | F | GACGAAGAGCCCTTACAGTTCCA | SEQ ID NO. 3 |
| | R | TCTGCAGTGCCTCCACTATG | SEQ ID NO. 4 |
| Col-I | F | CCTGGTCCCTCTGGAAATG | SEQ ID NO. 5 |
| | R | GGAAGCCTCTTTCTCCTCTC | SEQ ID NO. 6 |

2.7 Result Analysis

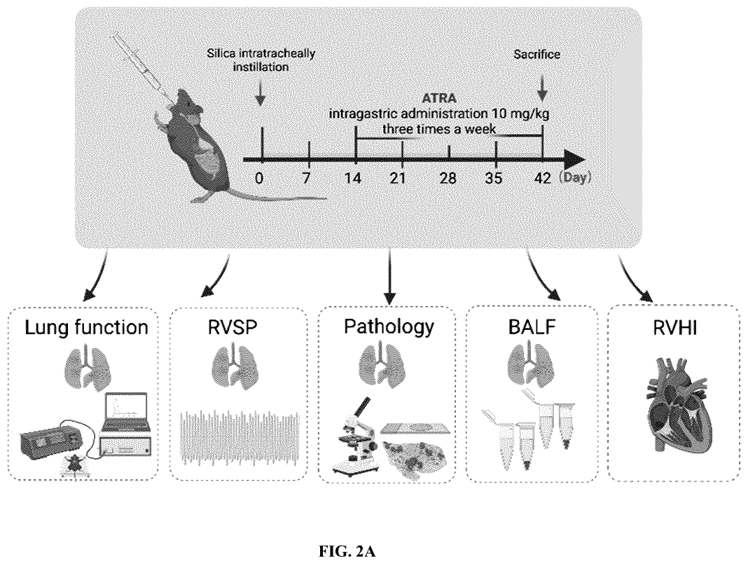

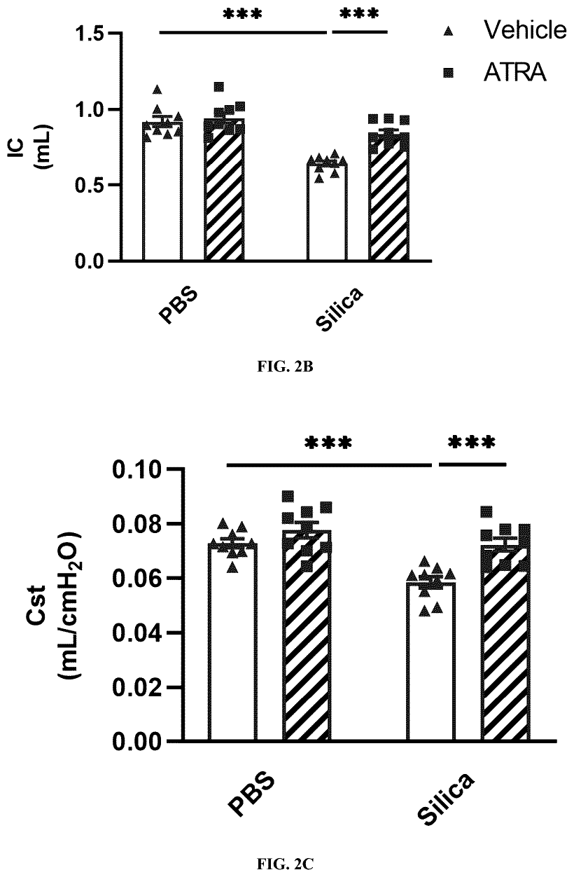

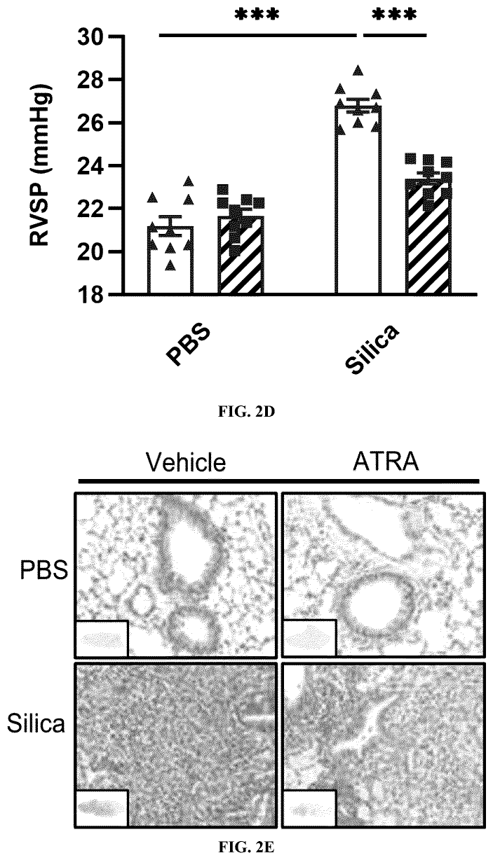

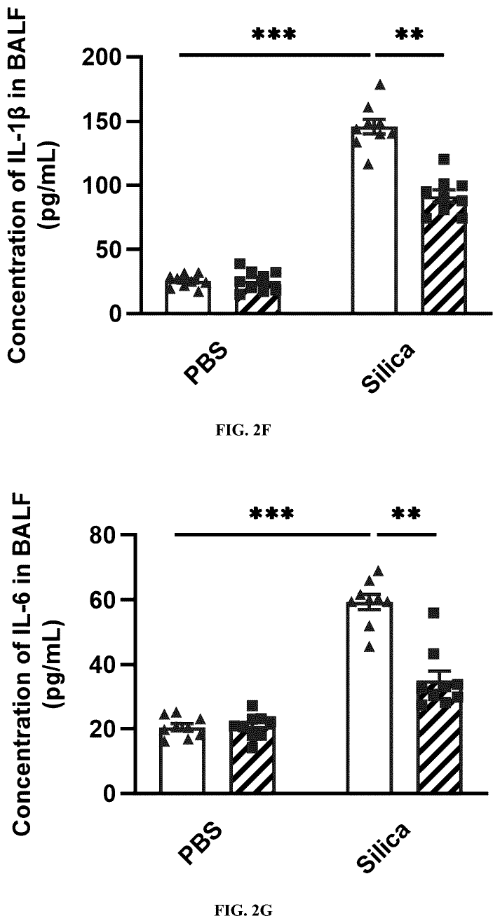

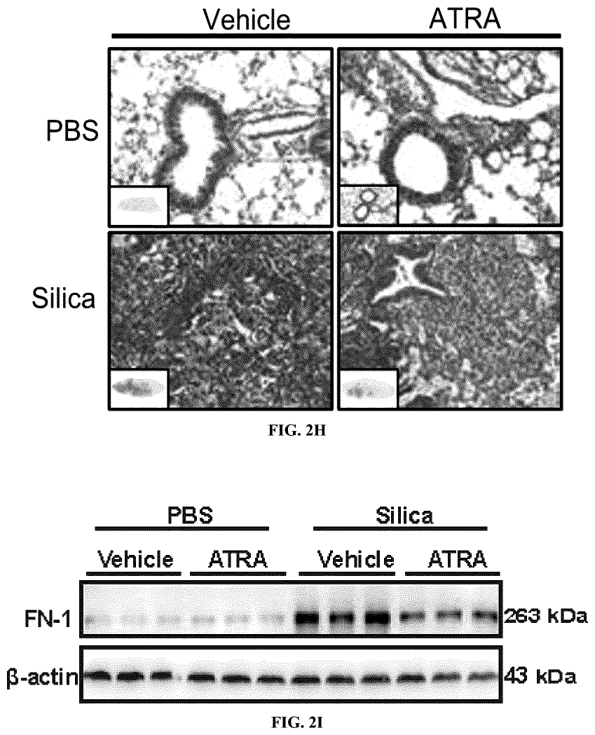

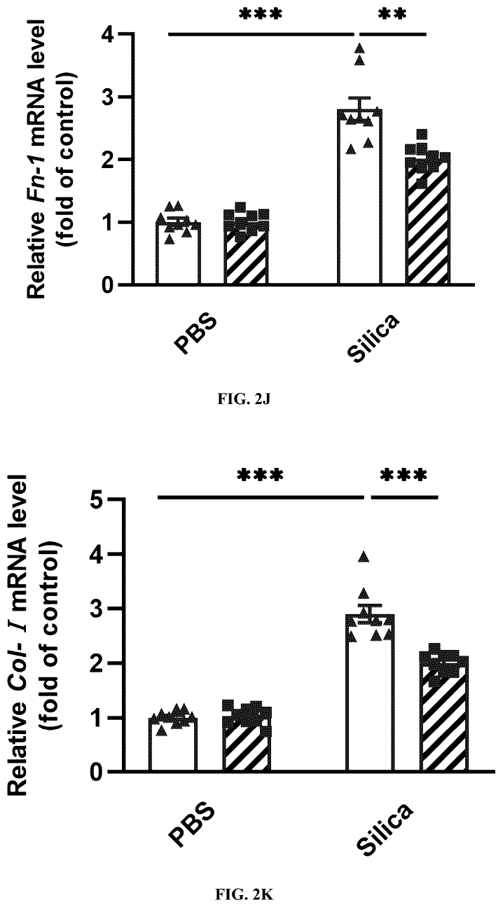

Treatment of mice with pneumoconiosis with ATRA could significantly improve their cardiopulmonary function, inflammation, and fibrosis (FIG. 2A). Compared with control mice, the IC (FIG. 2B) and the Cst (FIG. 2C) of ATRA-treated mice with pneumoconiosis were improved to a certain extent, and RVSP was further lowered to a certain extent (FIG. 2D). Pathological staining showed that ATRA could reduce the severity of inflammation and fibrosis (FIGS. 2E and 2H). ATRA further lowered concentrations of inflammatory factors IL-1β and IL-6 in the BALF of the mice with pneumoconiosis (FIGS. 2F and 2G) and reduced levels of fibrosis factors FN-1 and Col-I (FIGS. 21 to 2K).

### Example 3

VAD Aggravates Pneumoconiosis. 3.1 Construction of a Mouse Model of Pneumoconiosis and Corresponding Special Dietary Treatment.

Male C57BL/6J mice (aged 8-10 weeks and weighing 25-30 g) were selected and housed in an SPF grade laboratory animal room, and the model of pneumoconiosis was constructed by one-time intratracheal instillation of silica. The mice were divided into four groups (n=9). (1) PBS+VAS group: a vitamin A-sufficient diet (VAS diet) was provided. After the mice were fed with the VAS diet for 8 weeks, 40 μL of sterile PBS was administered by intratracheal instillation. (2) PBS+VAD group: a vitamin A-deficient diet (VAD diet) was provided. After the mice were fed with the VAD diet for 8 weeks, 40 μL of sterile PBS was administered by intratracheal instillation. (3) Si+VAS group: after the mice were fed with the VAS diet for 8 weeks, a silica suspension (300 mg/mL, 40 μL) was administered by intratracheal instillation. (4) Si+VAD group: after the mice were fed with the VAD diet for 8 weeks, a silica suspension (300 mg/mL, 40 μL) was administered by intratracheal instillation. All mice were sacrificed 6 weeks after modeling, and the corresponding samples were collected for detection.

The remaining experimental procedures were the same as 2.2, 2.3, 2.4, 2.5, and 2.6.

3.2 Result Analysis

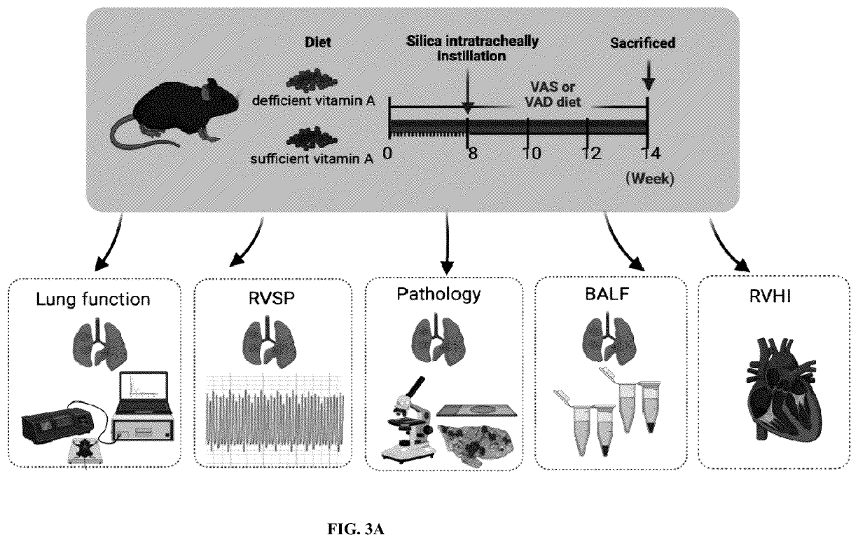

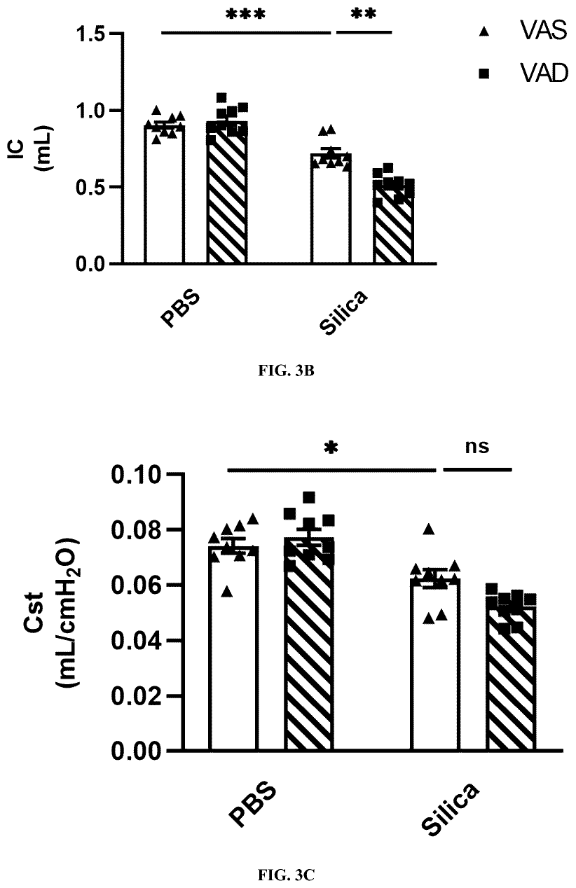

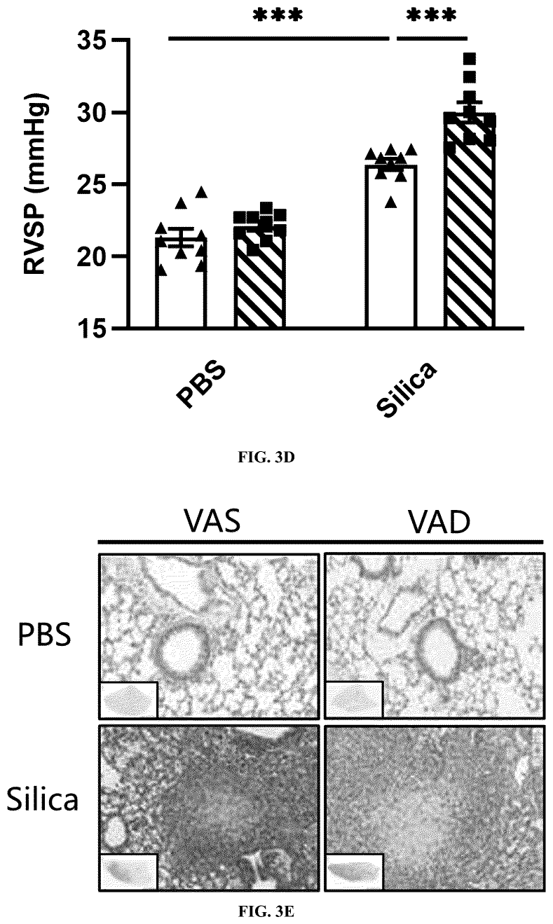

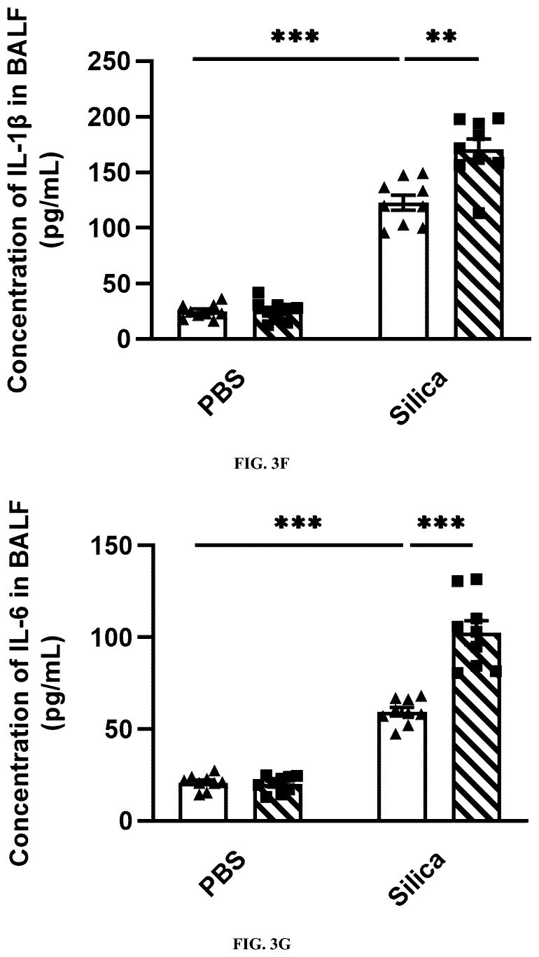

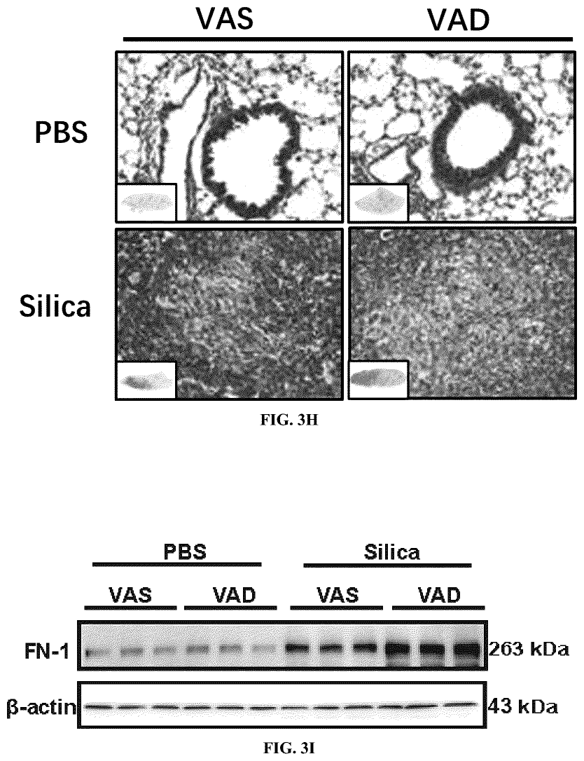

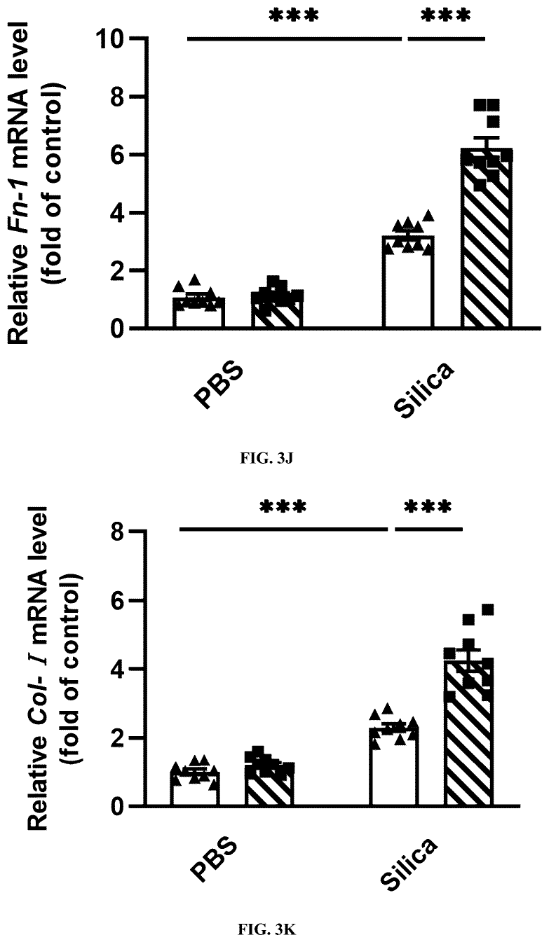

The mice with pneumoconiosis were intervened with a VAD diet to consume in vivo ATRA and aggravate the progression of pneumoconiosis (FIG. 3A). Compared with the VAS group fed with a normal diet, cardiopulmonary functions of the mice with pneumoconiosis were aggravated to varying degrees (FIG. 3B to 3D). Pathological staining also indicated that ATRA deficiency aggravated the severity of inflammation and fibrosis in the mice with pneumoconiosis (FIGS. 3E and 3H). Inflammatory factors, IL-1β and IL-6 (FIGS. 3F and 3G), as well as fibrosis factors, FN-1 and Col-I (FIGS. 3I to 3K), also increased in the mice with pneumoconiosis due to ATRA deficiency.

1. A method for treating pneumoconiosis, comprising:
administering retinol metabolites to a subject in need, three times a week, with a dose of 10 mg/kg for each administration, wherein the retinol metabolites are capable of lowering concentrations of inflammatory factors IL-1β and IL-6 in the subject and reducing levels of fibrosis factors FN-1 and Col-I of the subject.

2. The method according to claim 1, wherein the retinol metabolites comprise one or more of all-trans retinoic acid (ATRA), a precursor of ATRA, a metabolite of ATRA, and a modified product of ATRA.

3. (canceled)

4. (canceled)

5. The method according to claim 1, wherein the pneumoconiosis comprises silicosis, coal worker's pneumoconiosis, graphite pneumoconiosis, carbon black pneumoconiosis, asbestosis, talc pneumoconiosis, cement pneumoconiosis, mica pneumoconiosis, kaolin pneumoconiosis, aluminosis, arc-welders pneumoconiosis, and foundry workers' pneumoconiosis.
